(19) United States
(12) Patent Application Publication
(10) Pub. No.: US 2011/0251285 A1
(43) Pub. Date: Oct. 13, 2011

(54) COMBINATIONS OF PRESERVATIVES FOR OPHTHALMIC COMPOSITIONS

(75) Inventors: Walter L. Tien, Irvine, CA (US); Richard S. Graham, Irvine, CA (US); Ramakrishnan Srikumar, Aliso Viejo, CA (US)

(73) Assignee: ALLERGAN, INC., Irvine, CA (US)

(21) Appl. No.: 13/081,318

(22) Filed: Apr. 6, 2011

Related U.S. Application Data
(60) Provisional application No. 61/321,701, filed on Apr. 7, 2010.

(51) Int. Cl. A01N 37/52; A01P 1/00; A61P 27/04

(52) U.S. Cl. 514/635

(57) ABSTRACT

The present invention provides a preservative composition for protecting ophthalmic solutions from microbial attack comprising a combination of benzalkonium ion and polyhexamethylene biguanide (PHMB) wherein the combined concentrations of benzalkonium ion and polyhexamethylene biguanide (PHMB) in said composition is sufficient to provide protection against microbial attack when said composition is added to an ophtalmic solution as compared to said ophthalmic soulution having the same concentration of benzalkonium ion and polyhexamethylene biguanide (PHMB), alone.

### CROSS REFERENCE

This application claims the benefit of U.S. Provisional Application Ser. No. 61/321,701, filed Apr. 7, 2010 which is hereby incorporated by reference in its entirety.

### BACKGROUND OF THE INVENTION

The present invention relates to the field of preservatives for ophthalmic solutions and, in particular, aqueous ophthalmic solutions that are prone to spoilage by contact with the environment. More particularly, the invention relates to ophthalmic compositions, including those useful for drug delivery to the eye, and those to treat dry eye and otherwise care for the eye, contact lens care compositions and the like, which are benefited from being preserved.

### SUMMARY OF RELATED ART

Ophthalmic compositions often utilize at least one preservative, depending on the type of composition. Certain therapeutics included in such compositions are often irritating to the eye. This adverse effect can be minimized or eliminated in some cases if the preservative is present at a reduced concentration. In addition, such a reduced concentration of preservative may be advantageous in preventing other adverse effects that may be caused by certain preservatives. However, in some cases a reduced preservative concentration may produce a composition which does not pass certain standards such as the US Pharmacopeia APET criteria and European Pharmacopeia EP-A and/or EP-B preservative efficacy criteria.

Various ophthalmic compositions, such as solutions, emulsions and suspensions and the like, are used in association with administering therapeutics or therapeutic components to or through the eyes. For example, an oil-in-water emulsion may be used as a carrier for a therapeutic component to be administered to the eyes. Such compositions often benefit from being effectively preserved, for example, using preservatives and/or concentrations of preservatives which do not cause significant detrimental effect to the composition or to the human or animal to whom the composition is administered.

There is a need for preservatives which, when added to ophthalmic compositions, provide for an enhanced effect in the compositions thereby allowing for the use of reduced concentrations of preservatives which stabilize such compositions against microbial attack but do not cause detrimental effects, such as eye irritation.

### BRIEF SUMMARY OF THE INVENTION

The present invention provides a preservative composition for protecting ophthalmic solutions from microbial attack comprising a combination of benzalkonium ion and polyhexamethylene biguanide (PHMB), wherein the combined concentration of benzalkonium ion and polyhexamethylene biguanide (PHMB), in said composition, is sufficient to provide protection against microbial attack when said composition is added to an ophthalmic solution as compared to said ophthalmic soulution having the same concentration of benzalkonium ion or polyhexamethylene biguanide (PHMB), alone.

The present preservative composition may be used to provide an ophthalmic solution comprising a combination of benzalkonium ion and polyhexamethylene biguanide (PHMB), sufficient to protect said ophthalmic solution from microbial attack, wherein the same amount of benzalkonium ion and polyhexamethylene biguanide (PHMB), alone, is insufficient to protect said ophthalmic solution from microbial attack.

The present invention also provides, in an ophthalmic solution susceptible to microbial attack as a result of the concentration of a first preservative being insufficient to provide protection against said microbial attack, an ophthalmic solution which is not susceptible to microbial attack, by providing a second preservative at a concentration insufficient to provide protection against said microbial attack, alone, wherein said first preservative comprises a benzalkonium ion and said second preservative comprises polyhexamethylene biguanide (PHMB).

Finally, said invention provides a method of lowering the concentration of preservative required to protect an ophthalmic solution from microbial attack, which comprises providing a combination of preservatives, each in an amount insufficient to provide protection of said ophthalmic solution from microbial attack to obtain an ophthalmic solution that is not susceptible to microbial attack, wherein said combination of preservatives comprises benzalkonium ion and polyhexamethylene biguanide (PHMB).

The above concentrations may be determined empirically. That is, the skilled artisan can determine by experiment that a concentration of PHMB is ineffective in a given ophthalmic solution or composition to provide protection against microbial attack. Similarly, the skilled artisan can determine by experiment that a concentration of benzalkonium ion is ineffective in said given ophthalmic solution or composition to provide protection against microbial attack. Finally, the skilled artisan can determine that the combination of both PHMB and benzalkonium ion in the same ophthalmic solution or composition at the concentration where PHMB and benzalkonium ion, alone, is ineffective to provide protection against microbiological attack, provides protection against microbiological attack.

Said benzalkonium ion may be provided by benzalkonium chloride.

The above preservative composition is especially useful in preparing an ophthalmic solution as a multidose presentation and, for example, the solution may comprise 3 ppm of polyhexamethylene biguanide (PHMB) and 10 ppm benzalkonium chloride. When the solution is formulated with the above preservative composition said solution meets both the Ph Eur-B & A criteria.

Polyhexamethylene biguanide (PHMB) has the following structure:

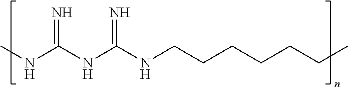

wherein n is an integer of from 7.5 to 13, preferably from 7.5 to 11.5.

Polyhexamethylene biguanide (PHMB) is a disinfectant and a preservative used for disinfection on skin and in cleaning solutions for contact lenses. It is a polymer or oligomer where biguanide functional groups are connected by hexyl hydrocarbon chains, with varying chain lengths. PHMB is specifically bactericidal at very low concentrations (10 mg/l) and is also fungicidal. It has a unique method of action in that the polymer strands are incorporated into the bacterial cell wall, which disrupts the membrane and reduces its permeability, which has a lethal effect to bacteria. It is also known to bind to bacterial DNA, alter its transcription, and cause lethal DNA damage. It has very low toxicity to higher organisms such as human cells, which have more complex and protective membranes. PHMB is a mixture of molecules of various sizes; different-sized molecules have a synergistic effect.

In one aspect of the invention said solution is an artificial tear solution and said solution is useful for treating keratitis sicca. In another aspect of the invention said solution is useful for treating elevated intraocular pressure.

### DETAILED DESCRIPTION OF THE PATENT

It has surprisingly been discovered that the use of a combination of benzalkonium chloride (BAK) and polyhexamethylene biguanide (PHMB), each at a sub-effective concentration, provides effective protection against microbial contamination, meets antimicrobial preservative effectiveness testing (APET) regulatory criteria and minimizes any ocular toxicity that may result from the use of higher concentrations of a single preservative. This invention is of particular importance for ophthalmic products, where corneal and ocular toxicity can interfere with the commercial success of a product.

It was surprisingly found that 3 ppm polyhexamethylene biguanide (PHMB), alone, failed to meet both Ph Eur-B & A criteria due to poor antimicrobial efficacy against fungi. But, the combination of 10 ppm of polyhexamethylene biguanide (PHMB) with 10 or 20 ppm BAK passed Ph Eur-B & A criteria. It was also found that 20 ppm of BAK resulted in a composition that failed to meet both Ph Eur-B & A criteria. But, the combination of 20 ppm BAK with 3 ppm of polyhexamethylene biguanide (PHMB) passed Ph Eur-B & A criteria.

In one aspect of the present invention, polyhexamethylene biguanide (PHMB) is present in an ophthalmic composition in a less then effective amount to aid in preserving the ophthalmic composition, for example, in an amount that is ineffective to preserve, one or more components of the composition. Preferably, polyhexamethylene biguanide (PHMB) is provided in such concentration so as to not substantially or significantly detrimentally affect the functioning of other components in the compositions, such as for example, a therapeutic component, e.g., a quinoxaline component, included in the composition.

In one embodiment, polyhexamethylene biguanide (PHMB) is employed in a concentration of about 0.01 ppm or more. For example, polyhexamethylene biguanide (PHMB) may be employed in an amount in a range of about 0.1 ppm to about 10 ppm, 0.1-5 ppm, 0.1-4.0 ppm, 0.1-3.0 ppm, 0.1-2.0 ppm, or 0.1-1.0 ppm. Preferably, polyhexamethylene biguanide (PHMB) is present in an amount in a range of about 1.0 ppm to about 3.0 ppm.

Very effective concentrations of polyhexamethylene biguanide (PHMB) in the present compositions are greater than about 1 ppm. Such concentrations are selected to be ineffective to preserve the compositions, alone, and do not detrimentally affect the other components of the compositions or cause significant detrimental effects to the human or animal to which the composition is administered. Such concentrations of polyhexamethylene biguanide (PHMB), together with a benzalkonium ion, as described elsewhere herein, provide preservative efficacy and acceptably long product shelf life. The other member of the combination of preservatives that constitute the present invention is benzalkonium ion, which is also provided in a sub-effective concentration.

Preferably, the benzalkonium ion is provided in such concentration so as to not substantially or significantly detrimentally affect the functioning of other components in the compositions, such as for example, a therapeutic component, e.g., a quinoxaline component, included in the composition. The benzalkonium ion is provided as a salt, e.g. a halide, and most preferably as the chloride, i.e. bezalkoniumchloride.

The benzalkonium ion is employed in a concentration of about 0.01 ppm or more. For example, the benzalkonium ion may be employed in an amount in a range of about 0.1 ppm to about 30 ppm. Preferably, the benzalkonium ion is present in an amount in a range of from about 1.0 ppm to about 20 ppm.

In one aspect of the present invention the inclusion of a borate component in the present compositions is provided. As disclosed in US Published Patent Application 20040191332, which is hereby incorporated by reference, a borate component is shown to be effective to enhance the effect of PHMB in ophthalmic compositions. For example, the borate component may enhance the antibacterial and/or antifungal activity of the above preservatives in the ophthalmic compositions. In one embodiment, the borate component prolongs the shelf life of a composition relative to a substantially identical composition without the borate component. The presently useful borate components include, without limitation, boric acid, salts of boric acid, and the like and mixtures thereof. Examples include, without limitation, borax, sodium tetraborate, sodium perborate, orthoboric acid, metaboric acid, mixtures thereof and the like. The present invention contemplates the use of any suitable boron-containing compound, for example, a boron-containing compound which is ophthalmically acceptable in the present compositions, which is effective to enhance the preservative efficacy of a composition in accordance with the present invention.

A borate component may be present in a composition in any amount which may be effective to enhance the effect of the benzalkonium ion and/or polyhexamethylene biguanide (PHMB) in the composition. In one embodiment, the borate component is employed in a composition in concentration of about 0.001% (w/v) or more. For example, the borate component may be employed in an amount in a range of about 0.001% to about 10% (w/v) or about 20% (w/v). In another example, the borate component may be employed in an amount in a range of about 0.005% to about 5% (w/v) or about 10% (w/v). In another example, the borate component may be employed in an amount in a range of about 0.005% or 0.01% to about 2% (w/v) or about 4% (w/v). Advantageously, the borate component is present in an amount in a range of about 0.01% to about 1% (w/v).

In another aspect of the present invention, as disclosed in US Published Patent Application 20040191332, a glycerin component, such as, without limitation, glycerin and the like and mixtures thereof, can also enhance an effect of the above preservatives in a composition. For example, a glycerin component can enhance an effect of polyhexamethylene biguanide (PHMB) in a composition when the composition also includes a borate component. The glycerin component may be present in a composition in any amount effective to enhance the effect polyhexamethylene biguanide (PHMB). For example, the glycerin component may enhance the antibacterial and/or antifungal activity of polyhexamethylene biguanide (PHMB) in a composition. In one embodiment, the glycerin component prolongs the shelf life of a composition relative to a substantially identical composition without the glycerin component. Glycerin components are very useful to enhance the preservative efficacy of ophthalmic compositions comprising emulsions having aqueous components and oily components.

In one embodiment, the glycerin component is employed in a composition in concentration of about 0.001% (w/v) or more. For example, the glycerin component may be employed in an amount in a range of about 0.001% to about 30% (w/v). The glycerin component may be employed in an amount in a range of about 0.005% or about 0.01% or about 0.1% to about 10% (w/v) or about 15% (w/v) or about 20% (w/v) or about 30% (w/v). Preferably, the glycerin component is present in an amount in a range of about 0.1% to about 5% (w/v).

In a further important aspect of the present invention, the present compositions are substantially free of certain carbohydrates and/or alcohols or sugar-alcohols (i.e., polyols). For example, a composition may be substantially free of mannitol, sorbitol, xylitol and the like and mixtures thereof. In one embodiment, polyhexamethylene biguanide (PHMB) is included in a composition that is substantially free of one or more certain carbohydrates, alcohols and/or polyols, as described elsewhere herein, and has one or more enhanced effects, preferably enhanced preservative efficacy, relative to a substantially identical composition which includes such substances, for example, which includes 1.5% (w/v) of one or more such carbohydrates, alcohols and/or polyols. In one particularly useful embodiment, a composition is substantially free of mannitol.

In one embodiment, the present compositions which are substantially free of mannitol have enhanced preservative efficacy relative to a substantially identical composition which includes 1.5% (w/v) of mannitol. In one embodiment, the preserved composition substantially free of mannitol has prolonged shelf life relative to a substantially identical composition which includes 1.5% (w/v) of mannitol.

In summary, regarding the carbohydrates and/or alcohols or sugar-alcohols (i.e., polyols), discussed above the compositions consist essentially of an ophthalmic solution comprising a combination of benzalkonium ion and polyhexamethylene biguanide (PHMB), sufficient to protect said ophthalmic solution from microbial attack, wherein the same amount of benzalkonium ion and polyhexamethylene biguanide (PHMB), alone, is insufficient to protect said ophthalmic solution from microbial attack and which solution may further comprise a borate and/or a glycerin component, as discussed above, and/or a therapeutic component, as discussed below.

A therapeutic component may be included in compositions of the present invention. Examples of useful therapeutic components include, but are not limited to, NMDA antagonists; antibacterial substances such as beta-lactam antibiotics, for example, cefoxitin, n-formamidoylthienamycin and other thienamycin derivatives, tetracyclines, chloramphenicol, neomycin, carbenicillin, colistin, penicillin G, polymyxin B, vancomycin, cefazolin, cephaloridine, chibrorifamycin, gramicidin, bacitracin and sulfonamides; aminoglycoside antibiotics such as gentamycin, kanamycin, amikacin, sisomicin and tobramycin; quinolones such as norfloxacin, ofloxacin and the like; nitrofurazones and analogs thereof; antihistaminics and decongestants such as pyrilamine, chlorpheniramine, tetrahydrazoline, antazoline and analogs thereof; mast-cell inhibitors of histamine release such as cromolyn and the like; anti-inflammatories such as cortisone, hydrocortisone, hydrocortisone esters, betamethasone, dexamethasone, dexamethasone sodium phosphate, prednisone, methylprednisolone, medrysone, fluorometholone, prednisolone, prednisolone sodium phosphate, triamcinolone, indainethacin, sulindac, and analogs thereof; miotics and anticholinergics such as echothiophate, pilocarpine, physostigmine salicylate, diisopropylfluorophosphate, epinephrine, dipivaloylepinephrine, neostigmine echothiopate iodide, demecarim bromide, carbamoyl choline chloride, methacholine, bethanechol and analogs thereof; mydriatics such as atrophine, homatropine, scopolamine, hydroxyamphetamine, ephedrine, cocaine, tropicamide, phenylephrine, cyclopentolate, oxyphenonium, eucatropine; and the like and mixtures thereof.

Other therapeutic components include, without limitation: antiglaucama drugs, for example, timolol, and especially its maleic salt and R-timolol, and combinations of timolol, timolol maleate and/or R-timolol with pilocarpine; adrenergic agonists and/or antagonists such as epinephrine and epinephrine complexes, and prodrugs such as bitartrate, borate, hydrochloride and dipivefrine derivatives; carbonic anhydrase inhibitors such as acetazolamide, dichlorphenamide, 2-(p-hydroxyphenyl)-thiothiophene-sulfonamide, 6-hydroxy-2-benzothiazolesulfonamide, and 6-pivaloyloxy-2-benzothiazolesulfonamide; antiparasitic compounds and/or anti-protozoal compounds such as ivermectin, pyrimethamine, trisulfapidimidine, clindamycin and corticosteroid preparations; compounds having antiviral activity such as acyclovir, 5-iodo-2′-deoxyuridine (IDU), adenosine arabinoside (Ara-A), trifluorothymidine, interferon, and interferon-inducing agents such as poly I:C; antifungal agents such as amphotericin B, nystatin, flucytosine, natamycin and miconazole; anesthetic agents such as etidocaine cocaine, benoxinate, dibucaine hydrochloride, dyclonine hydrochloride, naepaine, phenacaine hydrochloride, piperocaine, proparacaine hydrochloride, tetracaine hydrochloride, hexylcaine, bupivacaine, lidocaine, mepivacaine and prilocaine; ophthalmic diagnostic agents, such as: (a) those used to examine the retina, for example, sodium fluorescein, (b) those used to examine the conjunctiva, cornea and lacrimal apparatus, for example, fluorescein and rose bengal and (c) those used to examine abnormal pupillary responses, for example, methacholine, cocaine, adrenaline, atropine, hydroxyamphetamine and pilocarpine; ophthalmic agents used as adjuncts in surgery, for example, alpha-chymotrypsin and hyaluronidase; chelating agents, for example, ethylenediaminetetraacetic acid (EDTA), salts thereof, and deferoxamine; immunosuppressants and anti-metabolites, for example, methotrexate, cyclophosphamide, 6-mercaptopurine and azathioprine; and combinations of the agents mentioned above, such as antibiotics/antiinflammatories combinations, for example, the combination of neomycin sulfate and dexamethasone sodium phosphate, and combinations concomitantly used for treating glaucoma, for example, a combination of timolol maleate and aceclidine; and the like and mixtures thereof.

Other useful therapeutic components include ocular hypotensive agents such as disclosed in Woodward et al U.S. Pat. No. 5,688,819; pyranoquinolinone derivatives such as disclosed in Cairns, et al. U.S. Pat. No. 4,474,787; compounds having retinoid-like activities such as disclosed in Chandraratna U.S. Pat. No. 5,089,509; ketorolac/pyrrole-1-carboxylic acids such as disclosed in Muchowski, et al. U.S. Pat. No. 4,089,969; ofloxacins/benzoxazine derivatives such as disclosed in Hayakawa, et al. U.S. Pat. No. 4,382,892 and memantines such as disclosed in Lipton et al U.S. Pat. No. 5,922,773. The disclosure of each of U.S. Pat. Nos. 5,688,819; 4,474,787; 5,089,509; 4,089,969; 4,382,892; and 5,922,773 is incorporated herein in its entirety by reference.

In one useful embodiment, the present therapeutic components include adrenergic agonists. The adrenergic agonists may be amine-containing chemical entities with pKa's of greater than about 7, for example, in a range of about 7 (or greater than about 7) to about 9.

In one embodiment, the useful therapeutic components include alpha-adrenergic agonists. Examples of alpha-adrengergic agonists include, but are not limited to, adrafinil, adrenolone, amidephrine, apraclonidine, budralazine, quinoxalines, clonidine, cyclopentamine, detomidine, dimetofrine, dipivefrin, ephedrine, epinephrine, fenoxazoline, guanabenz, guanfacine, hydroxyamphetamine, ibopamine, indanazoline, isometheptene, mephentermine, metaraminol, methoxamine, methylhexaneamine, metizolene, midodrine, naphazoline, norepinephrine, norfenefrine, octodrine, octopamine, oxymetazoline, phenylephrine, phenylpropanolamine, phenylpropylmethylamine, pholedrine, propylhexedrine, pseudoephedrine, rilmenidine, synephrine, tetrahydrozoline, tiamenidine, tramazoline, tuaminoheptane, tymazoline, tyramine, xylometazoline, and the like and mixtures thereof. Other therapeutic agents include cyclosporine, bimatoprost and brimonidine.

In one useful embodiment, the therapeutic components include alpha-2-adrenergic agonists. As used herein, the term “alpha-2 adrenergic agonist” includes chemical entities, such as compounds, ions, complexes and the like, that may produce a net sympatholytic response, resulting in increased accommodation, for example, by binding to presynaptic alpha-2 receptors on sympathetic postganglionic nerve endings or, for example, to postsynaptic alpha-2 receptors on smooth muscle cells. A sympatholytic response is characterized by the inhibition, diminishment, or prevention of the effects of impulses conveyed by the sympathetic nervous system. The alpha-2 adrenergic agonists of the invention may bind to the alpha-2 adrenergic receptors presynaptically, causing negative feedback to decrease the release of neuronal norepinephrine. Additionally, they also may work on alpha-2 adrenergic receptors postsynaptically, inhibiting beta-adrenergic receptor-stimulated formation of cyclic AMP, which contributes to the relaxation of the ciliary muscle, in addition to the effects of postsynaptic alpha-2 adrenergic receptors on other intracellular pathways. Activity at either pre- or postsynaptic alpha-2 adrenergic receptors may result in a decreased adrenergic influence. Decreased adrenergic influence results in increased contraction resulting from cholinergic innervations. Alpha-2 adrenergic agonists also include compounds that have neuroprotective activity. For example, 5-bromo-6-(2-imidozolin-2-ylamino) quinoxaline is an alpha-2-adrenergic agonist which has a neuroprotective activity through an unknown mechanism.

Without limiting the invention to the specific groups and compounds listed, the following is a list of representative alpha-2 adrenergic agonists useful in this invention: imino-imidazolines, including clonidine, apraclonidine; imidazolines, including naphazoline, xymetazoline, tetrahydrozoline, and tramazoline; imidazoles, including detomidine, medetomidine, and dexmedetomidine; azepines, including B-HT 920 (6-allyl-2-amino-5,6,7,8 tetrahydro-4H-thiazolo[4,5-d]-azepine and B-HT 933; thiazines, including xylazine; oxazolines, including rilmenidine; guanidines, including guanabenz and guanfacine; catecholamines and the like.

Particularly useful alpha-2-adrenergic agonists include quinoxaline components. In one embodiment, the quinoxaline components include quinoxalines, derivatives thereof and mixtures thereof. The derivatives of quinoxaline include, without limitation, (2-imidozolin-2-ylamino) quinoxalines, salts thereof and mixtures thereof. In one embodiment, the derivatives of quinoxaline include 5-halide-6-(2-imidozolin-2-ylamino) quinoxalines, salts thereof and mixtures thereof. The “halide” of the 5-halide-6-(2-imidozolin-2-ylamino) quinoxalines may be a fluorine, a chlorine, an iodine, or preferably, a bromine, to form 5-bromo-6-(2-imidozolin-2-ylamino) quinoxaline (brimonidine), also known as brimonidine.

Other useful quinoxalines and quinoxaline derivatives are well known. For example, useful quinoxalines and derivatives of a quinoxaline include the ones disclosed by U.S. Pat. No. 5,021,416; U.S. Pat. No. 5,703,077; and U.S. Pat. No. 3,890,319. The disclosure of each of these three patents is incorporated in its entirety by reference herein.

The quinoxaline and derivatives thereof, for example, brimonidine, are amine-containing and preferably have pKa's of greater than about 7, preferably about 7.5 to about 9.

Analogs, salts, for example, ophthalmically acceptable salts and other derivatives of the foregoing chemical entities that function in a similar manner to provide a desired therapeutic effect also are specifically contemplated for use as therapeutic components in the present compositions.

In one useful embodiment, the amount of therapeutic component in the present composition is in the range of about 0.01% to about 30% (w/v). The amount of therapeutic component may be in the range of about 0.1% (w/v) to about 10% (w/v). For example, the amount of therapeutic component may be in the range of about 0.1% (w/v) to about 0.6% (w/v). In one embodiment, the therapeutic component is an adrenergic agonist and is present in the composition in the range of about 0.1% (w/v) to about 0.6% (w/v), for example, about 0.15% (w/v).

The present compositions may conveniently be presented as solutions or suspensions in aqueous liquids or non-aqueous liquids, or as oil-in-water or water-in-oil liquid emulsions. The present compositions may include one or more ingredients which are conventionally employed in compositions of the same general type.

The present compositions may be in the form of aqueous suspensions, oily suspensions and oil-in-water emulsions as disclosed in US Published Patent Application 20040191332.

The carrier component of the present compositions is ophthalmically acceptable. A carrier component or other material is “ophthalmically acceptable” when it is substantially compatible with ocular tissue. That is, it does not cause significant or undue detrimental effects when brought into contact with ocular tissue. Preferably, the ophthalmically acceptable material is also substantially compatible with other components of the present compositions. The carrier component may include one or more components which are effective in providing such ophthalmic acceptability and/or otherwise benefiting the composition and/or the eye to which the composition is administered and/or the patient whose eye is being treated. Advantageously, the carrier component is aqueous-based, for example, comprising a major amount, that is at least about 50% by weight, of water.

Examples of suitable materials useful in the present carrier components include water, mixtures of water and water-miscible solvents such as lower alkanols or aralkanols, oily components, vegetable oils, polyalkylene glycols, petroleum-based jelly, ethyl cellulose, ethyl oleate, polyvinylpyrrolidone, isopropyl mirstate, other conventionally employed ophthalmically acceptable materials and the like and mixtures thereof.

The carrier component may also include auxiliary substances such as emulsifiers, wetting agents, bodying agents, buffer components, acids and/or bases, tonicity adjuster components, surfactant components, viscosity agents, lubricity components, preservative components, other materials useful in ophthalmic formulations and the like, including but not limited to, such substances which are conventionally used in ophthalmic compositions.

Examples of optionally useful bodying agents include, but are not limited to, various polyethylene glycols, carbowaxes, petroleum jelly and the like.

Suitable buffers include, but are not limited to, inorganic buffers such as phosphate buffers, borate buffers and the like, and organic buffers, such as acetate buffers, citrate buffers, tromethamine, and the like.

Tonicity adjusters optionally useful in the present compositions include, but are not limited to, dextrose, potassium chloride and/or sodium chloride and the like, preferably sodium chloride.

Acids optionally useful in the present compositions include boric acid, hydrochloric acid, acetic acid, other acids which are ophthalmically acceptable in the concentrations used, and the like.

Bases which may be included in the present compositions include, but are not limited to, sodium and/or potassium hydroxides, other alkali and/or alkaline earth metal hydroxides, organic bases, other bases which are ophthalmically acceptable in the concentrations used, and the like.

The acid/bases/buffers preferably are included, if at all, to provide and/or maintain the present compositions at a pH in the physiologically acceptable range, more preferably in a range of about 4 to about 8.5, still more preferably about 6 to about 8, and especially about 6.8 to about 8.

Surfactant components optionally useful in the compositions of the present invention include, but are not limited to, lipoprotein detergents that when present in the compositions reduce the surface tension between the compositions and the eye (lacrimal) fluid. Preferably, nonionic surfactants are used.

Viscosity agents optionally useful in the compositions of the present invention include, but are not limited to, cellulose derivatives such as hydroxypropylmethyl cellulose, carboxymethyl cellulose, hydroxyethyl cellulose, other viscosity inducing materials useful in ophthalmic formulations, and the like.

In one embodiment of the invention, the present compositions include a polyanionic component. Advantageously, the polyanionic component is present in an amount effective to provide lubrication to an eye when the composition is administered to the eye. The polyanionic component is often present in an amount of at least about 0.1% w/v of the composition. For example, the polyanionic component may be present in an amount in a range of about 0.1% or about 0.2% to about 1% (w/v) or 5% (w/v) or about 10% (w/v) of the composition. In another example, the polyanionic component is present in an amount in a range of about 0.6% to about 1.8% (w/v) of the composition. The polyanionic components are disclosed in US Published Patent Application 20040191332.

Also included within the scope of this invention are preserved compounds which increase in viscosity upon administration to the eye. For example, “gelling polysaccharides” which are disclosed in U.S. Pat. No. 5,212,162 which is incorporated in its entirety herein by reference. Also disclosed in this patent are ophthalmic formulations containing carrageenans and furcellarans which are administered as partially gelled liquids which gel upon instillation into the eye. Additionally, U.S. Pat. Nos. 4,136,173, 4,136,177, and 4,136,178, disclose the use of therapeutic compositions containing xanthan gum and locust bean gum which are delivered in liquid form to the eye and which gel upon instillation. U.S. Pat. No. 4,861,760 discloses ophthalmological compositions containing gellan gum which are administered to the eye as non-gelled liquids and which gel upon instillation. The disclosure of each of these four patents is incorporated in its entirety herein by reference.

Also within the scope of this invention are preserved oils, ointments, gels and the like. The present compositions may include components, such as cyclodextrins, to enhance the solubility of one or more other components included in the compositions. For example, steroids, which are hydrophobic, often exhibit an increase in water solubility of one order of magnitude or more in the presence of cyclodextrins. Any suitable cyclodextrin component may be employed in accordance with the present invention. The useful cyclodextrin components include, but are not limited to, those materials which are effective in increasing the apparent solubility, preferably water solubility, of poorly soluble active components and/or enhance the stability of the active components and/or reduce unwanted side effects of the active components. Examples of useful cyclodextrin components include, but are not limited to: .alpha.-cyclodextrin, derivatives of .alpha.-cyclodextrin, .beta.-cyclodextrin, derivatives of .beta.-cyclodextrin, .gamma.-cyclodextrin, derivatives of .gamma.-cyclodextrin, carboxymethyl-.beta.-cyclodextrin, carboxymethyl-ethyl-.beta.-cyclodextrin, diethyl-.beta.-cyclodextrin, dimethyl-.beta.-cyclodextrin, methyl-.beta.-cyclodextrin, random methyl-.beta.-cyclodextrin, glucosyl-.beta.-cyclodextrin, maltosyl-.beta.-cyclodextrin, hydroxyethyl-.beta.-cyclodextrin, hydroxypropyl-.beta.-cyclodextrin, sulfobutylether-.beta.-cyclodextrin, and the like and mixtures thereof. As used herein, the term “derivative”, as it relates to a cyclodextrin, means any substituted or otherwise modified compound which has the characteristic chemical structure of a cyclodextrin sufficiently to function as a cyclodextrin component, for example, to enhance the solubility and/or stability of active components and/or reduce unwanted side effects of the active components and/or to form inclusive complexes with active components, as described herein.

One or more additional components can be included in the present compositions based on the particular application for which the compositions are formulated. For example, the present compositions can be formulated to include a therapeutic component to be administered to the eyes.

The present preserved compositions may be administered to the eyes. These compositions, formulated appropriately, may be used in place of prior conventional compositions. For example, the compositions may be use in administering a therapeutic component to the eyes. In one embodiment, an antibiotic is administered to the eyes in a composition of the invention. In another example, the compositions of the invention may be used as a surgical irrigant.

The present compositions may also be used in the care of a contact lens, for example, to make wearing the lens safe and comfortable. The present compositions, formulated appropriately, may be used in conventional contact lens care regimens by using the present compositions in place of prior conventional compositions. In many instances, these contact lens care regimens involve contacting the lens with the present composition in an amount, and at conditions, effective to obtain the beneficial or desired contact lens care result.

The following non-limiting examples illustrate certain aspects of the present invention.

Each formulation set forth in the following examples is prepared by blending together the listed components in a conventional manner.

Each of these formulations is tested by performing an abbreviated preservative efficacy test using test organisms S. aureus, P. aeruginosa, c. albicans, E. coli and/or A. niger. The formulations are tested against United States Pharmacopeia Preservative Efficacy Test (USP), European Pharmacopeia Efficacy Test-A (EP-A) and European Pharmacopeia Efficacy Test-B (EP-B) criteria as indicated. Ten (10) ml of each formulation is challenged with approximately 10.sup.5 cfu/ml of test organism. At appropriate time intervals, the amount of bacterial and fungal survivors are assayed using Dey Engley broth (DE) as the neutralizer media. DE, along with filtration, is sufficient at neutralizing the antimicrobial agents in the compositions. One (1) ml of each sample is diluted into nine (9) ml of DE. One (1) ml of the 1:10 dilution is filtered through a 0.45 .mu.m filter and washed with 100 ml of a saline/polysorbate 80 solution. After washing the filtrate a second time with 100 ml of saline/polysorbate 80 solution, the filtrate is placed onto a TSA plate for bacteria and SAB for fungi.

The present invention is not to be limited in scope by the exemplified embodiments, which are only intended as illustrations of specific aspects of the invention. Various modifications of the invention, in addition to those disclosed herein, will be apparent to those skilled in the art by a careful reading of the specification, including the claims, as originally filed. It is intended that all such modifications will fall within the scope of the appended claims.

What is claimed is:

1. A preservative composition for protecting ophthalmic solutions from microbial attack comprising a combination of benzalkonium ion and polyhexamethylene biguanide (PHMB), wherein the combined concentrations of benzalkonium ion and PHMB, in said composition is sufficient to provide protection against microbial attack when said composition is added to an ophtalmic solution as compared to said ophthalmic solution having the same concentration of benzalkonium ion or PHMB, alone.

2. The solution of claim 1 wherein said benzalkonium ion is provided by benzalkonium chloride.

3. The solution of claim 1, wherein said PHMB has the following structure: 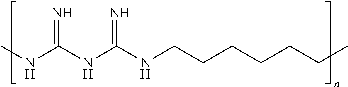
wherein n is an integer of from 7.5 to 13.

4. The solution of claim 1 comprising from 0.1 to 10 ppm PHMB and 0.1 to 30 ppm benzalkonium ion.

5. The solution of claim 3 comprising 3 ppm PHMB and 10 ppm benzalkonium ion.

6. The solution of claim 1 wherein said solution is a multidose presentation.

7. The solution of claim 5 wherein said solution is an artificial tear solution.

8. An ophthalmic solution comprising a combination of benzalkonium ion and PHMB, sufficient to protect said ophthalmic solution from microbial attack, wherein the same amount of benzalkonium ion and polyhexamethylene biguanide (PHMB), alone, is insufficient to protect said ophthalmic solution from microbial attack.

9. The solution of claim 8 wherein said benzalkonium ion is provided by benzalkonium chloride.

10. The solution of claim 8, wherein said PHMB has the following structure: 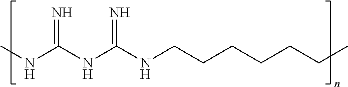
wherein n is an integer of from 7.5 to 13.

11. The solution of claim 8 comprising from 0.1 to 10 ppm PHMB and 0.1 to 30 ppm benzalkonium ion.

12. The solution of claim 11 comprising 3 ppm PHMB and 10 ppm benzalkonium ion.

13. The solution of claim 8 wherein said solution is a multidose presentation.

14. The solution of claim 8 wherein said solution is an artificial tear solution.

15. A method of lowering the concentration of preservative required to protect an ophthalmic solution from microbial attack, which comprises providing a combination of preservatives, each in an amount insufficient to provide protection of said ophthalmic solution from microbial attack to obtain an ophthalmic solution that is not susceptible to microbial attack, wherein said combination of preservatives comprises benzalkonium ion and PHMB.

16. The solution of claim 15 wherein said benzalkonium ion is provided by benzalkonium chloride.

17. The solution of claim 15, wherein said PHMB has the following structure: 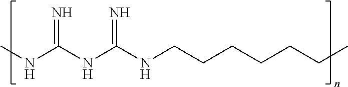
wherein n is an integer of from 7.5 to 13.

18. The solution of claim 16 comprising from 0.1 to 10 ppm PHMB and 0.1 to 30 ppm benzalkonium ion.

19. The solution of claim 18 comprising 3 ppm PHMB and 10 ppm benzalkonium ion.

20. The solution of claim 15 wherein said solution is a multidose presentation.

21. The solution of claim 17 wherein said solution is an artificial tear solution.
